(19) United States
(12) Patent Application Publication
(10) Pub. No.: US 2013/0345403 A1
(43) Pub. Date: Dec. 26, 2013

(54) AQUEOUS FACTOR VIII SOLUTION

(71) Applicants: Christian Rischel, Copenhagen S (DK); Christina Jespersgaard, Copenhagen V (DK); Are Bogsnes, Nivaa (DK); Janus Krarup, Gentofte (DK)

(72) Inventors: Christian Rischel, Copenhagen S (DK); Christina Jespersgaard, Copenhagen V (DK); Are Bogsnes, Nivaa (DK); Janus Krarup, Gentofte (DK)

(73) Assignee: NOVO NORDISK A/S, Bagsvaerd (DK)

(21) Appl. No.: 13/994,268

(22) PCT Filed: Nov. 30, 2011
(86) PCT No.: PCT/EP2011/071339

§ 371 Date: Aug. 28, 2013

Related U.S. Application Data
(60) Provisional application No. 61/424,389, filed on Dec. 17, 2010.

(30) Foreign Application Priority Data
Dec. 16, 2010 (EP) 10195288.5

(51) Int. Cl. C07K 14/755

(52) U.S. Cl. CPC C07K 14/755
USPC 530/383; 435/68.1

(57) ABSTRACT

The present invention relates to methods for stabilizing FVIII in aqueous solutions comprising a relatively high concentration of FVIII. The invention furthermore relates to such aqueous solutions as well as use thereof.

The present invention relates to the field of methods for improving Factor VIII yields. In particular, the present invention relates to methods and buffer compositions/aqueous solutions useful for reducing Factor VIII aggregate formation/precipitation.

### BACKGROUND

FVIII/Factor VIII is a large, complex glycoprotein that is used in haemophilia A therapy/prophylaxis either in a plasma derived form or in the form of a recombinant protein that may optionally be post-translationally modified by e.g. chemical and/or enzymatic methods.

It is generally associated with difficulties to keep large proteins in solution at a high concentration as they tend to form aggregates. It is well known that FVIII (with or without the B domain) has poor solubility compared to most other proteins. Visible precipitation can occur at concentrations as low as 15 μg/ml, invisible precipitation occurs at much lower concentrations, which is particularly undesirable in connection with e.g. posttranslational modification of the protein where it is desirable to keep the FVIII concentration well above 1 μg/ml. Keeping FVIII at a high concentration can also be desirable in connection with e.g. storage and/or purification of FVIII. Finally, it is difficult to obtain high yields of rFVIII expressed in mammalian cell lines, even when expressed as a B-domain deleted/truncated variant, and it is therefore highly desirable to take measures to reduce the amount of aggregate formation of rFVIII in order to minimize the loss-of-yield associated with FVIII precipitation in solutions having a FVIII concentration of at least 0.5 μg/ml.

There is no suggestion in the prior as to how in vitro aggregate formation of FVIII can be reduced. In WO09108806, a salt concentration of 250 mM NaCl is used in an elution step in connection with purification of FVIII after post-translational modification.

### SUMMARY

The present invention relates to a method of stabilizing FVIII in an aqueous solution having an FVIII concentration of at least 1 μg/ml and a pH of 5.5-8.5, wherein said method comprises keeping FVIII in an aqueous solution comprising salt at a concentration of at least 300 mM and glycerol at a concentration of 5-30%. The present invention furthermore relates to such solutions as well as use thereof.

It is shown herein by the inventors that this combination of ingredients can reduce the tendency of FVIII to precipitate under conditions of relatively high FVIII-concentrations. The methods and solutions of the present invention are also useful in connection with situations where the FVIII concentration is lower than 0.5 μg/ml such as e.g. in connection with concentration and/or purification of FVIII where the concentration will be increased to at least 0.5 μg/ml.

### DESCRIPTION OF THE INVENTION

“FVIII/Factor VIII” is a large, complex glycoprotein that primarily is produced by hepatocytes. Human FVIII consists of 2351 amino acids, including signal peptide, and contains several distinct domains, as defined by homology. There are three A-domains, a unique B-domain, and two C-domains. The domain order can be listed as NH2-A1-A2-B-A3-C1-C2-COOH. FVIII circulates in plasma as two chains, separated at the B-A3 border. The chains are connected by bivalent metal ion-bindings. The A1-A2-B chain is termed the heavy chain (HC) while the A3-C1-C2 is termed the light chain (LC). “FVIII” is herein understood to be plasma derived or recombinant FVIII, wt FVIII or any FVIII variant having FVIII activity in e.g. a chromogenic assay. Examples of such FVIII variants include B-domain truncated/deleted variants, and/or FVIII conjugated to one or more side groups (e.g. PEG, other water soluble polymers, fatty acid derivatives, Fc:FVIII fusions) and/or FVIII variants having one or more amino acid modifications in one or more of the A and/or C domains, etc. One or more of such FVIII modifications may result in an increased circulatory half life the FVIII variant as compared to wt FVIII.

“B domain”: The length of the B domain in the wt FVIII molecule is about 907 amino acids. The length of the B domain in B domain truncated FVIII molecules/variants may vary from about 10 to about 800 amino acids, such as e.g. from about 10 amino acids to about 700 acids, such as e.g. about 12-500 amino acids, 12-400 amino acids, 12-300 amino acids, 12-200 amino acids, 15-100 amino acids, 15-75 amino acids, 15-50 amino acids, 15-45 amino acids, 20-45 amino acids, 20-40 amino acids, or 20-30 amino acids. The truncated B-domain may comprise fragments of the heavy chain and/or the light chain and/or an artificially introduced sequence that is not found in the wt FVIII molecule. The terms “B-domain truncated” and “B-domain deleted” may be used interchangeably herein.

“Ionic Strength/I” of a solution is a well known measure of the concentration of ions in that solution. The ionic strength, I, of a solution is a function of the concentration of all ions present in that solution. Table 1 converts molar concentrations of various salts that can be used in connection with the present invention into ionic strength.

TABLE 1
Ionic strength (I) as a function of different compositions

| | NaCl, KCl, | |
|---|---|---|
| | NH₄Ac, NaAc, | CaCl₂, CaAc₂, |
| | KAc, NH₄Cl | MgCl₂, MgAc₂, |
| 10 mM | 10 | 30 |
| 30 mM | 30 | 90 |
| 50 mM | 50 | 150 |
| 100 mM | 100 | 300 |
| 300 mM | 300 | 900 |
| 500 mM | 500 | 1500 |
| 1000 mM | 1000 | 3000 |

“Aqueous solution”/“aqueous buffer” is herein understood to be a solution where water is the primary solvent and wherein the solution comprises either no organic solvents or insignificant amounts and/or trace amounts of organic solvents, such as e.g. less than 1% organic solvents.

“Salt” is herein understood to be any salt, e.g. one or more of the salts according to table 1.

“Glycerol” in the context of the present invention means glycerol as well as other compounds that may replace glycerol such as e.g. polyols, such as e.g. ethylene glycol, propylene glycol, erythritol, mannitol, sorbitol, xylitol, 1,3-propane diol, diethanolamine, sucrose, dextrose, trehalose, glucose. It is well known to the man skilled in the art that this type of compounds can replace glycerol in connection with stabilisation of FVIII in an aqueous solution.

“Detergent/surfactant” is herein meant to include any detergent/surfactant, e.g. one or more of the following detergents: SDS, Triton X-100, X114, CHAPS, DOC, NP-40, Tween 80, and Tween 20.

Divalent cations are added to the solutions according to the present invention, e.g. Mg2+, Cu2+, Zn2+, Ca2+ “Ca2+” can be added in the form of one or more of the salts listed in table 1 as well as CaOH2.

“Size exclusion chromatography/SEC/gel-filtration chromatography” is a chromatographic method in which molecules in solution are separated based on their size (more correctly, their hydrodynamic volume). Typically, when an aqueous solution is used to transport the sample through the column, the technique is known as gel-filtration chromatography, versus the name Gel permeation chromatography, which is used when an organic solvent is used as a mobile phase. SEC is a widely used polymer characterization method because of its ability to provide good Mw results for polymers. The main application of gel-filtration chromatography is the fractionation of proteins and other water-soluble polymers, while gel permeation chromatography is used to analyze the molecular weight distribution of organic-soluble polymers.

“Post-translational modification of FVIII”: is herein meant to be any modification of rFVIII or plasma derived FVIII such as e.g. conjugation of the molecule with hydrophilic polymers (e.g. poly ethylene glycol (PEG)), fatty acid derivates, albumin, Fc domains, etc. The modification/conversion of FVIII may take place using e.g. chemical and/or enzymatic approaches. One example of a method for enzymatic post-translational modification of peptides is disclosed in WO03031464.

“Stabilization of FVIII” is herein meant to be a reduction of the loss of active FVIII. In connection with storage, purification, and post-translational modification of FVIII under conditions with a relatively high FVIII concentration, a major cause of loss of FVIII yield is “aggregation/precipitation” of FVIII molecules. “Stabilization” can herein thus be viewed as reduction of precipitation of FVIII in high concentration FVIII solutions. In the Examples, it is demonstrated how the solutions and/or methods according to the present invention result in a reduction in the loss of FVIII yield.

### LIST OF EMBODIMENTS

### Embodiment 1

In a first aspect, the present invention thus relates to a method of stabilizing FVIII in an aqueous solution having an FVIII concentration of at least 1 μg/ml and a pH of 5.5-8.5, wherein said method comprises keeping FVIII in an aqueous solution comprising salt at a concentration of at least 300 mM, glycerol at a concentration of 5-35%, divalent cation at a concentration of 2-20 mM (preferably Ca²⁺), and a detergent at a concentration of 0.05-0.3 g/kg.

### Embodiment 2

The FVIII concentration of the method according to any of the embodiments can be at least about 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 50, 75, 100, 125, 150, 175, 200, 225, 250, 275, 300, 350, 400, 450, 500, 550, 600, 650, 700, 750, 800, 850, 900, 1000, 2000, 3000, 4000, 5000, 6000, 7000, 8000, 9000, 10,000, 15,000, 20,000, or 25,000 μg/ml.

### Embodiment 3

The FVIII concentration of any embodiment according to the present invention can be in the range of e.g. 1-25,000 μg/ml, such as e.g. 1-20,000 μg/ml, 1-15,000 μg/ml, 1-10,000 μg/ml, 1-5000 μg/ml, 1-4000 μg/ml, 1-3000 μg/ml, 1-2000 μg/ml, 1-1000 μg/ml, 1-900 μg/ml, 1-800 μg/ml, 1-700 μg/ml, 1-600 μg/ml, 1-500 μg/ml, 1-400 μg/ml, 1-300 μg/ml, 1-200 μg/ml, 1-100 μg/ml, 5-5000 μg/ml, 5-4000 μg/ml, 5-3000 μg/ml, 5-2000 μg/ml, 5-1000 μg/ml, 5-900 μg/ml, 5-800 μg/ml, 5-700 μg/ml, 5-600 μg/ml, 5-500 μg/ml, 5-400 μg/ml, 5-300 μg/ml, 5-200 μg/ml, 5-100 μg/ml, 10-25,000 μg/ml, 10-20,000 μg/ml, 10-15,000 μg/ml, 10-10,000 μg/ml, 10-5000 μg/ml, 10-4000 μg/ml, 10-3000 μg/ml, 10-2000 μg/ml, 10-1000 μg/ml, 10-900 μg/ml, 10-800 μg/ml, 10-700 μg/ml, 10-600 μg/ml, 10-500 μg/ml, 10-400 μg/ml, 10-300 μg/ml, 10-200 μg/ml, 10-100 μg/ml, 15-25,000 μg/ml, 15-20,000 μg/ml, 15-10,000 μg/ml, 15-5000 μg/ml, 15-4000 μg/ml, 15-3000 μg/ml, 15-2000 μg/ml, 15-1000 μg/ml, 15-900 μg/ml, 15-800 μg/ml, 15-700 μg/ml, 15-600 μg/ml, 15-500 μg/ml, 15-400 μg/ml, 15-300 μg/ml, 15-200 μg/ml, 15-100 μg/ml, 20-5000 μg/ml , 20-4000 μg/ml, 20-3000 μg/ml, 20-2000 μg/ml 20-1000 μg/ml, 20-900 μg/ml, 20-800 μg/ml, 20-700 μg/ml, 20-600 μg/ml, 20-500 μg/ml, 20-400 μg/ml, 20-300 μg/ml, 20-200 μg/ml, or 20-100 μg/ml.

### Embodiment 4

A method according to any of the embodiments of the present invention, wherein the salt is a monovalent salt selected from the groups consisting of: one or more sodium salt and/or one or more an ammonium salt. Examples of such salts are listed in table 1.

### Embodiment 5

A method according to any one of the embodiments according to the present invention, wherein the salt is NaCl.

### Embodiment 6

A method according to any one of the embodiments according to the invention, wherein the salt concentration in the aqueous solution is from 275-1500 mM, such as e.g. 275-1400 mM, 275-1300 mM, 275-1200 mM, 275-1100 mM, 275-100 mM, 275-1000 mM, 275-900 mM, 275-800 mM, 275-700 mM, 275-600 mM, 275-500 mM, 275-400 mM 300-1500 mM, 300-1400 mM, 300-1300 mM, 300-1200 mM, 300-1100 mM, 300-1000 mM, 300-900 mM, 300-800 mM, 300-700 mM, 300-600 mM- 300-500 mM, 300-400 mM, 325-1500 mM, 325-1400 mM, 325-1300 mM, 325-1200 mM, 325-1100 mM, 325-1000 mM, 325-900 mM, 325-800 mM, 325-700 mM, 325-600 mM, 325-500 mM, 325-400 mM, 350-1500 mM, 350-1400 mM, 350-1300 mM, 350-1200 mM, 350-1100 mM, 350-1000 mM, 350-900 mM 350-800 mM, 350-700 mM, 350-600 mM, 350-500 mM, 350-400 mM, 400-1500 mM, 400-1400 mM, 400-1300 mM, 400-1200 mM, 400-1100 mM, 400-1000 mM, 400-900 mM, 400-800 mM, 400-700 mM, 400-600 mM, 400-500 mM, 450-1500 mM, 450-1400 mM, 450-1300 mM, 450-1200 mM, 450-1100 mM, 450-1000 mM, 450-900 mM, 450-800 mM, 450-700 mM, 450-600 mM, 500-1500 mM, 500-1400 mM, 500-1300 mM, 500-1200 mM, 500-1100 mM, 500-1000 mM, 500-900 mM, 500-800 mM, 500-700 mM, or 500-600 mM.

### Embodiment 7

A method according to any one of the embodiments according to the invention, wherein FVIII is a B domain truncated variant.

### Embodiment 8

A method according to any one of the embodiments according to the invention, where the glycerol concentration is from 5-35%, such as e.g. 5-30%, 5-25%, 5-20%, 5-15%, 5-10%, 12.5-35%, 12.5-30%, 12.5-25%, 12.5-20%, 12.5-15%, 15-35%, 15-30%, or 15-20% (W/W).

### Embodiment 9

A method according to any one of the embodiments according to the invention, where the concentration of the divalent cation is from 2-20 mM, such as e.g. 2-15 mM, 2-10 mM, 2-5 mM, 5-20 mM, 5-15 mM, 5-10 mM, 10-20 mM, or 10-15 mM. Divalent cations can be added in the form of e.g. the calcium salts listed in table 1.

### Embodiment 10

A method according to any one of the embodiments according to the invention, wherein the detergent concentration is from 0.05-0.5 g/kg, such as e.g. 0.05-0.4 g/kg, 0.05-0.3 g/kg, 0.05-0.2 g/kg, 0.05-0.1 g/kg, 0.1-0.5 g/kg, 0.1-0.4 g/kg, 0.1-0.3 g/kg, or 0.1-0.2 g/kg. Examples of detergents suitable for use in connection with the present invention include SDS, Triton X-100, X114, CHAPS, DOC, NP-40, Tween 80, and Tween 20.

### Embodiment 11

A method according to any one of the embodiments according to the invention, wherein pH of the solution is from 5.5-8.5, such as e.g. 5.5-8.0, 5.5-7.5, 5.5-7.0, 5.5-6.5, 5.5-6.0, 6.0-8.5, 6.0-8.0, 6.0-7.5, 6.0-7.0, 6.0-6.5, 6.5-8.5, 6.5-8.0, 6.5-7.5, 6.5-7.0, 7.0-8.5, 7.0-8.0, 7.0-7.5, 7.5-8.5, 7.5-8.0, or 8.0-8.5.

### Embodiment 12

A method according to any one of the embodiments according to the invention, wherein the Fill molecule is a B domain truncated variant, the FVIII concentration is at least 1 μg/ml, the salt concentration is about 500 mM, the glycerol concentration is 10-20%, the concentration of the divalent cation is about 10 mM, the Tween concentration is 0.1-0.2 g/kg and pH of the solution is from 6-8.

### Embodiment 12

An aqueous FVIII solution comprising at least 1 μg FVIII/ml, a pH of 5.5-8.5, salt at a concentration of at least 300 mM, glycerol at a concentration of 5-30%, divalent cation at a concentration of 2-20 mM (preferably Ca²⁺), and a detergent at a concentration of 0.05-0.3 g/kg. The detergent is preferably Tween 20.

### Embodiment 13

A FVIII solution according to any one of the embodiments according to the present invention, wherein the salt is a monovalent salt selected from the groups consisting of: a sodium salt or an ammonium salt.

### Embodiment 14

A FVIII solution according to any one of the embodiments according to the present invention, wherein the salt is NaCl.

### Embodiment 15

A FVIII solution according to any one of the embodiments according to the invention, wherein the salt concentration in the aqueous solution is from 300-1000 mM. Preferably the salt is NaCl.

### Embodiment 16

A FVIII solution according to any one of the embodiments according to the invention, wherein the Fill molecule is a B domain truncated variant, the FVIII concentration is at least 1 μg/ml, the salt concentration is about 500 mM, the glycerol concentration is 10-20%, the Ca2+concentration is about 10 mM, the Tween concentration is 0.1-0.2 g/kg and pH of the solution is from 6-8.

### Embodiment 17

A FVIII solution according to any one of the embodiments according to the invention may furthermore comprise a FVIII concentration as set forth in connection with embodiment 2, a salt concentration as set forth in embodiment 6, a glycerol concentration as set forth in embodiment 8, a concentration of divalent cations as set forth in embodiment 9, a concentration of detergents as set forth in embodiment 10, and a pH as set forth in embodiment 11. The specific salt, can be selected from any of the alternatives as suggested herein. The specific source of divalent cations can likewise be selected from any of the alternatives suggested herein. The specific source of detergent can likewise be selected from any of the alternatives suggested herein.

### Embodiment 18

A method for size exclusion chromatographic separation or purification of FVIII, wherein FVIII is stabilized during separation or purification using a method according to any one of the embodiments of the present invention and/or a solution according to any one of the embodiments of the present invention.

### Embodiment 19

A method for post-translational modification of FVIII, wherein FVIII is stabilized during the modification process using a method according to any one of the embodiments according to the present invention and/or a solution according to any one of the embodiments of the invention.

### Embodiment 20

Use of a solution according to any one of the embodiments of the present invention and/or a method according to any one of the embodiments of the present invention for stabilizing FVIII.

### Embodiment 21

A method of stabilizing FVIII in an aqueous solution having an FVIII concentration of at least 1 μg/ml and a pH of 5.5-8.5, wherein said method comprises keeping FVIII in an aqueous solution comprising, salt at a concentration of at least 300 mM, and glycerol at a concentration of 5-30%.

### Embodiment 22

A method according to any of the embodiments of the present invention, wherein said aqueous solution comprises a divalent cation at a concentration of 2-20 mM.

### Embodiment 23

A method according to any of the embodiments of the present invention wherein the divalent cation is MgCl₂.

### Embodiment 24

A method according to any of the embodiments of the present invention, wherein the divalent cation is CaCl₂.

### Embodiment 25

A method according to any one of the embodiments of the present invention, wherein said aqueous solution comprises a detergent at a concentration of 0.05-0.3 g/kg. The detergent is preferably Tween.

### Embodiment 26

An aqueous FVIII solution comprising at least 1 μg FVIII/ml, a pH of 5.5-8.5, salt at a concentration of at least 300 mM, and glycerol at a concentration of 5-30%.

### Embodiment 27

A solution according to any of the embodiments of the present invention, wherein said solution further comprises a detergent at a concentration of 0.05-0.3 g/kg. The detergent is preferably Tween.

### Embodiment 28

A solution according to any one of the embodiments of the present invention, wherein said solution further comprises a divalent cation at a concentration of 2-20 mM.

### Embodiment 29

A solution according to any of the embodiments of the present invention wherein the divalent cation is MgCl₂.

### Embodiment 30

A solution according to any of the embodiments of the present invention, wherein the divalent cation is CaCl₂.

### EXAMPLES

### Example 1

A FVIII solution was buffer exchanged to 10 mM HEPES, 0.5 M NaCl, 20% (v/v) glycerol, 2 mM CaCl₂, 0.02% tween80, pH 7.5 and concentrated to about 30 mg/ml. A 96-well microtiter plate for protein crystallisation was set up with buffers in the following pattern:

Rows (final concentrations after mixing with Factor VIII):
A. 50 mM Na acetate, pH 5.0,
B. 50 mM His, pH 5.5,
C. 50 mM His, pH 6.0,
D. 50 mM Imidazole, pH 6.5,
E. 50 mM Imidazole, pH 7.0
F. 50 mM HEPES, pH 7.5
G. 50 mM HEPES, pH 8.0
H. 50 mM Gly-gly, pH 9.0

Columns:
1 and 7: 0 M NaCl
2 and 8: 0.08 M NaCl
3 and 9: 0.2 M NaCl
4 and 10: 0.33 M NaCl
5 and 11: 0.53 M NaCl
6 and 12: 0.8 M NaCl

All wells contained 20% glycerol, 0.02% tween80. Columns 1-6 contained 2 mM CaCl2, columns 7-12 contained 7-12 16 mM CaCl2. 200 nl FVIII solution+400 nl buffer solution was combined in a droplet, the plate was sealed with transparent film, incubated for 24 hours and examined under a microscope. The droplets were scored for precipitation on the following scale: None: Clear, Low: Weak precipitate, High: heavy precipitate. The results are shown in the following table:

TABLE 2
Precipitation in 600 nanoliter drops of 10 mg/ml Factor VIII at different conditions,
as observed under a microscope.

| | 2 mM CaCl₂ | | | | | | 16 mM CaCl₂ | | | | | |
|---|---|---|---|---|---|---|---|---|---|---|---|---|
| | 0M | 0.08M | 0.2M | 0.33M | 0.53M | 0.8M | 0M | 0.08M | 0.2M | 0.33M | 0.53M | 0.8M |
| | NaCl | NaCl | NaCl | NaCl | NaCl | NaCl | NaCl | NaCl | NaCl | NaCl | NaCl | NaCl |
| pH 5.0 | High | High | High | High | High | High | High | High | High | High | High | Low |
| pH 5.5 | High | High | High | None | None | None | High | High | Low | None | None | None |
| pH 6.0 | High | High | None | None | None | None | High | High | None | None | None | None |
| pH 6.5 | High | High | Low | None | None | None | High | High | None | None | None | None |
| pH 7.0 | High | Low | None | None | None | None | High | None | None | None | None | None |
| pH 7.5 | Low | None | None | None | None | None | High | None | None | None | None | None |
| pH 8.0 | None | None | None | None | None | None | None | None | None | None | Low | None |
| pH 9.0 | High | High | High | Low | Low | None | None | None | None | None | None | None |

It is seen that concentrations of NaCl of 0.33 M and above (rows 4-6, 10-12) are most favourable for avoiding precipitation, in particular at the lowest values of pH.

### Example 2

A solution of Factor VIII was buffer exchanged to 10 mM HEPES, 0.5 M NaCl, 20% (v/v) glycerol, 10 mm CaCl2, 0.02% tween80, pH 7.5 and concentrated to 19 mg/ml on an amicon spinfilter. A 384-well microtiter plate was set up with the following pattern:

Rows (final concentrations after mixing with Factor VIII):
A. 50 mM His, pH 5.5,
B. 50 mM His, pH 6.0,
C. 50 mM Imidazole, pH 6.5,
D. 50 mM Imidazole, pH 7.0
E. 50 mM HEPES, pH 7.5
F. 50 mM HEPES, pH 8.0

Columns:
1: 0 M NaCl
2: 0.17 M NaCl
3: 0.23 M NaCl
4: 0.3 M NaCl
5: 0.4 M NaCl
6: 0.5 M NaCl
   All wells contained 20% glycerol, 0.02% tween 80 and 10 mM CaCl2. Buffer solutions and Factor VIII were mixed, with a final Factor VIII concentration of 5 mg/ml. The intensity of light scattering from each well was measured in a Wyatt DynePro plate reader. Higher intensity indicates higher self-association. The results are shown in the following table:

TABLE 3
Intensity of scattered light (normalized count rate) from 5 mg/ml FVIII under different
values of pH and NaCl concentration.
Normalized intensity

| | pH 5.5 | pH 6 | pH 6.5 | pH 7 | pH 7.5 | pH 8 |
|---|---|---|---|---|---|---|
| 0.17M NaCl | 4.98E+08 | 1.01E+09 | 5.07E+08 | 2.91E+09 | 1.27E+09 | 1.18E+09 |
| 0.23M NaCl | 1.53E+09 | 9.86E+08 | 1.67E+09 | 7.52E+08 | 6.15E+08 | 5.02E+08 |
| 0.3M NaCl | 1.22E+09 | 3.27E+09 | 8.34E+08 | 4.44E+08 | 3.67E+08 | 4.24E+08 |
| 0.4M NaCl | 8.27E+08 | 1.92E+09 | 3.18E+08 | 2.12E+08 | 2.63E+08 | 3.05E+08 |
| 0.5M NaCl | 1.52E+09 | 4.65E+08 | 2.16E+08 | 1.55E+08 | 1.63E+08 | 1.92E+08 |

It is seen that the lowest intensitites, which indicate a low degree of self-association, are found at high concentrations of NaCl.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### Example 3

UF/DF
2150 g of a N8 comprising solution was pH and CaCl₂ adjusted to 6.13 and a total of 10 mmol/kg, respectively. The N8 comprising solution was subsequently concentrated to approximately 4 mg/ml by ultra-filtration and then buffer exchanged by dia-filtration with 5 volumes of buffer containing: 20 mmol/kg histidine, 9 mmol/kg HCl, 0.5 mol/kg NaCl, 10 mM/kg CaCl₂, 20% glycerol pH 6.16. The N8 comprising solution was then further concentrated to 9.54 mg/ml. The yield was in the range of 97-98% dependent on the method of analysis. The level of total HMWP measured after completion of the concentration is depicted in the table below. In conclusion, this demonstrates the lack of HMWP formation despite a significant increase in FVIII concentration under conditions of high NaCl concentrations.

TABLE 4

| Sample descripton | HMWP (%) | Dimer (%) | Total HMWP (%) |
|---|---|---|---|
| N8 starting material | <0.3 | <0.3 | <0.3 |
| N8 UF/DF | <0.3 | <0.3 | 0.4 |

### Example 4

PEGylation of FVIII

The starting material was a solution containing 7.5 mg/ml FVIII in 0.5 M sodium chloride, 10 mM calciumchloride, 20% glycerol, 20 mM histidine and 9 mM hydrochloric acid resulting in a pH of 6.1. 210 ml of this solution was added 1.3 mg Sialidase, 42 mg ST3Gal1 and 1.7 g 40K PEG, and left to react for 17.7 hours at ambient room temperature. There were no signs of turbidity or precipitation at the end of the reaction.

### Example 5

Hydrophobic Interaction Chromatography in Flowtrough Mode of a FVIII Molecule

The purpose of this step was to remove an enzyme (ST3Gal3), used for sialylation of a FVIII molecule covalently modified with a 40K polyethyleneglycol group, and HMWP (high molecular weight protein) by means of hydrophobic interaction chromatography. A column, 0.5 cm in diameter, was packed to a bed height of 10.5 cm with TSK Phenyl 5PW resin, resulting in a bed volume of 2.1 ml. The column was equilibrated with 5 column volumes of a buffer consisting of 450 mM sodium chloride, 10 mM calciumchloride, 10% glycerol, 0.02% polysorbate 80, 20 mM histidine and 9 mM hydrochloric acid resulting in a pH of 6.1 and a conductivity of ˜35 mS/cm. The load, comprising the FVIII molecule at a concentration of 1.05 mg/ml and 0.025 mg/ml ST3Gal3, was added sodium chloride to reach the same conductivity (35 mS/cm) as the equilibration buffer, and histidine and hydrochloric acid to adjust to pH 6.1. The load (37.5 ml) was passed over the column followed by equilibration buffer. The purified FVIII product, which did not bind to the column, was collected in the flowthrough, resulting in 41.1 ml at a concentration of 0.85 mg/ml. The yield was 88.7%. The content of high molecular weight protein was reduced from 1.5% to 1.0%. ST3Gal3 was reduced from ˜24000 ppm to 1328 ppm, corresponding to a ˜18 fold reduction.

### Example 5

SEC Example

Size exclusion chromatography was performed on an reaction mixture mixture containing both rFVIIIa which is covalently attached to 40K PEG and its reactants (rFVIII and PEG) using an AKTA explorer and a BPG10 collumn packed with 1.8 L (10 h×23.5 cm h) of Superdex 200 from GE Healthcare. The flow rate was 0.8 CV/hr (4.24 ml/min), temperature was 22 deg C., the running buffer consisted of:

| | | | | |
|---|---|---|---|---|
| L-Histidine | 5.8 | g/kg | 37.4 | mmol/kg |
| 37% HCl | 0.7 | g/kg | 7.1 | mmol/kg |
| CaCl2 2H20 | 0.97 | g/kg | 6.6 | mmol/kg |
| L-Methionine | 0.21 | g/kg | 1.4 | mmol/kg |
| NaCl | 34.9 | g/kg | 597 | mmol/kg |
| Sucrose | 11.6 | g/kg | 33.9 | mmol/kg |
| Polysorbate 80 | 10 | g/kg | | |

Before loading, the column was cleaned using 1 CV of sodium hydroxide, equliibrated with 1.2 CV of buffer before auto zeroing the UV.

The column was loaded with 92 ml (approximately 5% of CV) of reaction mixture, having a concentration of 1.05 mg/ml (97 mg total).

A pool was collected by when the UV absorbance signal exceeded 0.15 AU/cm, yielding a pool volume of 202 ml with a concentration of 0.46 mg/ml, resulting in a yield of 98%.

The described size exclusion chromatography step is used to reduce process enzymes as well as other contaminants. The process enzyme ST3Gal3 was reduced 330-fold by the SEC step (from approximately 1328 ppm to 4 ppm).

1. A method of stabilizing FVIII in an aqueous solution having an FVIII concentration of at least 1 μg/ml and a pH of 5.5-8.5, wherein said method comprises keeping FVIII in an aqueous solution comprising salt at a concentration of at least 300 mM and glycerol at a concentration of 5-30%.

2. A method according to claim 1, wherein said aqueous solution comprises a divalent cation at a concentration of 2-20 mM.

3. A method according to claim 1, wherein said aqueous solution comprises a detergent at a concentration of 0.05-0.3 g/kg.

4. A method according to claim 1, wherein the salt is NaCl.

5. A method according to claim 1, wherein the salt concentration in the aqueous solution is from 300-1000 mM.

6. A method according to claim 1, wherein FVIII is a B domain truncated variant, the FVIII concentration is at least 1 μg/ml, the salt concentration is about 500 mM, the glycerol concentration is 10-20%, the concentration of the divalent cation is about 10 mM, the detergent concentration is 0.1-0.2 g/kg and pH of the solution is from 6-8.

7. An aqueous FVIII solution comprising at least 1 μg FVIII/ml, a pH of 5.5-8.5, salt at a concentration of at least 300 mM, and glycerol at a concentration of 5-30%.

8. A solution according to claim 7, wherein said solution further comprises a detergent at a concentration of 0.05-0.3 g/kg.

9. A solution according to claim 7, wherein said solution further comprises a divalent cation at a concentration of 2-20 mM.

10. A FVIII solution according to claim 7, wherein the salt is NaCl.

11. A FVIII solution according to claim 7, wherein the salt concentration in the aqueous solution is from 300-1000 mM.

12. A FVIII solution according to claim 7, wherein FVIII is a B domain truncated variant, the FVIII concentration is at least 1 μg/ml, the salt concentration is about 500 mM, the glycerol concentration is 10-20%, the divalent cation concentration is about 10 mM, the detergent concentration is 0.1-0.2 g/kg and pH of the solution is from 6-8.

13. A method for size exclusion chromatographic separation or purification of FVIII, wherein FVIII is stabilized during separation or purification using the method according to claims 1 and a solution according to any one of claims 7-12.

14. A method for post-translational modification of FVIII, wherein FVIII is stabilized during the modification process using the method according to claims 1 and an aqueous FVIII solution comprising at least 1 μg FVIII/ml, a pH of 5.5-8.5, salt at a concentration of at least 300 mM, and glycerol at a concentration of 5-30%.

15. A method for stabilizing FVIII comprising keeping FVII in an aqueous solution according to claim 7.
